(19) United States
(12) Patent Application Publication
(10) Pub. No.: US 2015/0025472 A1
(43) Pub. Date: Jan. 22, 2015

(54) COVER FOR A SYRINGE AND METHOD OF MAKING

(71) Applicant: Robert Dax Colwell, Seattle, WA (US)

(72) Inventor: Robert Dax Colwell, Seattle, WA (US)

(21) Appl. No.: 14/206,542

(22) Filed: Mar. 12, 2014

Related U.S. Application Data
(60) Provisional application No. 61/777,740, filed on Mar. 12, 2013.

(51) Int. Cl. A61M 5/31; A61M 5/00

(52) U.S. Cl. CPC A61M 5/3129; A61M 5/002; A61M 2005/3125
USPC 604/189; 29/428

(57) ABSTRACT

A medical assembly includes a syringe and a cover wrapped around the syringe. The syringe is configured to hold and dispense a medicine therein. The cover includes information pertaining to the medicine. The cover is movable between a rolled configuration and an unrolled configuration. The cover includes a re-sealable device to removably couple the cover to itself when in the rolled configuration. The cover includes radiation blocking material to protect the medicine. A method of attaching the cover to the syringe is provided.

### CROSS-REFERENCE TO RELATED APPLICATION

This application claims the benefit under 35 U.S.C. §119(e) of U.S. Provisional Patent Application No. 61/777,740, filed Mar. 12, 2013, the contents of which are incorporated herein by reference in their entirety.

### BACKGROUND

1. Technical Field

The present invention is directed to medical assemblies, and more particularly, to covers for syringes.

2. Description of the Related Art

It is important that medicines and other compounds are used appropriately by patients. In particular, at-home syringes and other applicators are prone to improper usage and/or improper dosage of the medicine due to lack of information or training. Typically, information pertaining to the medicine and the syringe is provided separate from the syringe itself, thereby posing a risk for improper use of the medicine and/or the syringe.

### BRIEF SUMMARY

A medical assembly may be summarized as including a syringe and a cover attached to and wrapped around the syringe. The cover may be movable between a rolled configuration and an unrolled configuration. The cover may contain dosage information for proper use of a medicine or other compound contained in the syringe.

The syringe includes a longitudinal body having an outer surface and a chamber. The chamber is configured to hold a medicine therein to be dispensed by the syringe. The cover includes a first edge portion and a second edge portion. The first edge portion is attached to the outer surface of the longitudinal body. In the rolled configuration, the cover is wrapped around the outer surface and the second edge portion is removably coupled to the cover itself or to the longitudinal body, or to both. The cover includes a main sheet extending between the first and second edge portions. The main sheet may have information pertaining to use of the medicine and the syringe. The information may be accessible when the cover is in the unrolled configuration and/or the rolled configuration.

In some aspects, the cover includes particular, advantageous features. At least a portion of the cover may be comprised of material to prevent radiation from passing through the cover to the compound contained in the chamber. Additionally, the first edge portion may include a perforated portion to remove the cover completely from the syringe. The second edge portion may include a re-sealable device to removably attach the cover when moved from the unrolled configuration to the rolled configuration. The information on the main sheet may include at least one of dosage information, compound information, instructional information, and brand information.

A method of making a medical assembly may be summarized as including providing a syringe having a longitudinal body with an outer surface and a chamber having a medicine therein. The method includes coupling a cover to the longitudinal body, such as the cover having the features described in the present disclosure. The method may include removably attaching the cover to itself when moving the cover to a rolled configuration. The method includes providing information on the cover that is accessible when the cover is in an unrolled configuration. The information may include dosage information. The cover may be fabricated to include radiation blocking material to block harmful rays from damaging or altering the medicine contained in the chamber.

### BRIEF DESCRIPTION OF THE SEVERAL VIEWS OF THE DRAWINGS

FIG. 1 is a perspective view of a medical assembly having a cover and a syringe, showing the cover in a rolled configuration according to one aspect of the present disclosure.

FIG. 2 is a perspective view of the medical assembly of FIG. 1 in which the cover is an unrolled configuration according to one aspect of the present disclosure.

FIG. 3 is a partially exploded view of a container assembly for holding a medical assembly, according to one embodiment.

FIG. 4 is a perspective view of the container assembly of FIG. 3, showing the container assembly in a closed configuration.

### DETAILED DESCRIPTION

The following detailed description is directed toward covers for syringes and methods of making the same. The description and corresponding figures are intended to provide an individual of ordinary skill in the art with enough information to enable that individual to make and use embodiments of the invention. Such an individual, however, having read this entire detailed description and reviewed the figures, will appreciate that modifications can be made to the illustrated and described embodiments, and/or elements removed therefrom, without deviating from the spirit of the invention. It is intended that all such modifications and deviations fall within the scope of the invention, to the extent they are within the scope of the associated claims.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

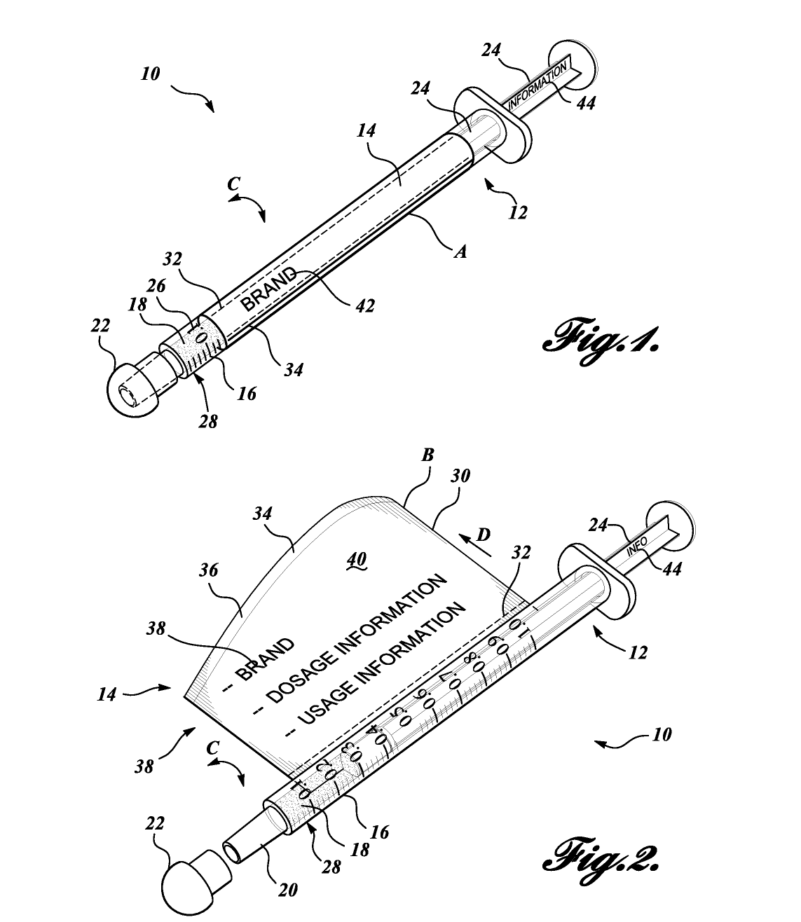

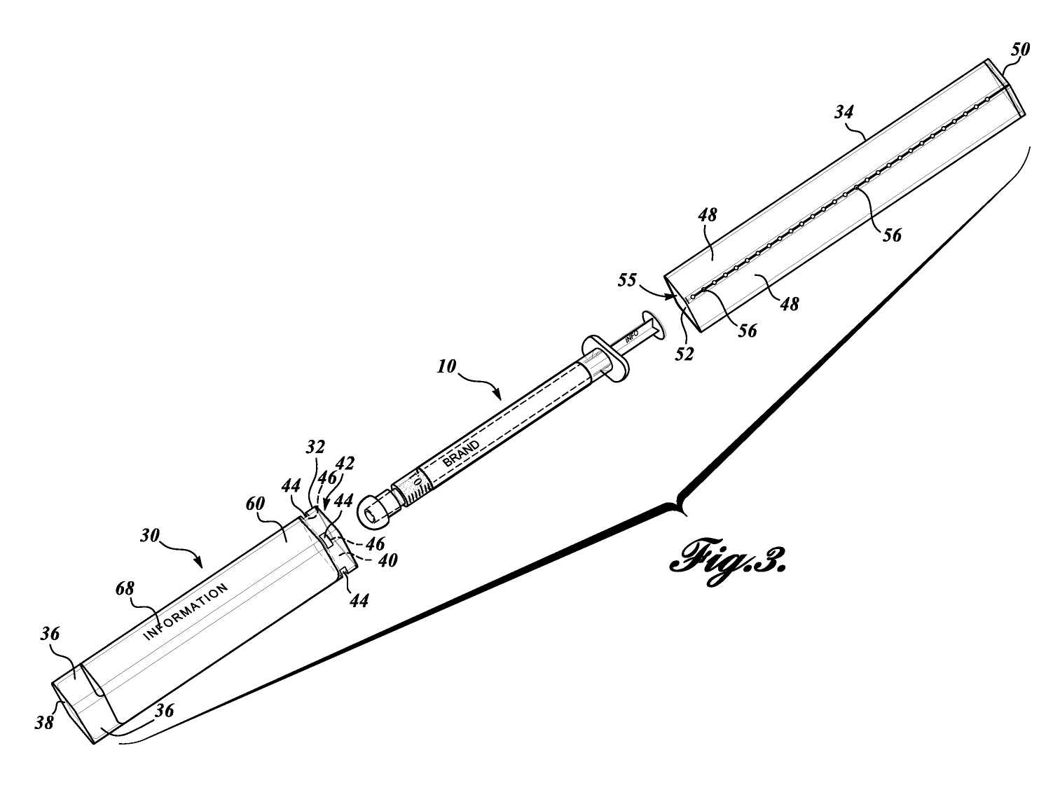

FIGS. 1 and 2 show a medical assembly 10 comprising a syringe 12 and a cover 14 according to one aspect. The cover 14 is movable between a rolled configuration A and an unrolled configuration B, as depicted by arrows C. The cover 14 may be re-sealable to itself when moved to the rolled configuration for reuse of the cover 14.

The syringe 12 includes a longitudinal body 16, a chamber 18, a tip 20, a cap 22, and a plunger 24. The chamber 18 is configured to hold contents therein, such as a medical compound 26, and the syringe 12 is configured to dispense the medical compound 26 out of the tip 20. The longitudinal body 16 includes an outer surface 28. The cover 14 includes a main sheet 30 having a first edge portion 32 and a second edge portion 34. As shown in FIG. 2, the illustrated first edge portion 32 is removably attached to the outer surface 28 of the longitudinal body 16. Accordingly, the first edge portion 32 can include a perforated edge to allow removal of the cover 14 from the syringe 12, as exemplified by Arrow D. The second edge portion 34 includes a re-sealable device 36, such as an adhesive, to re-seal the cover 14 to the syringe 12 when moving from the unrolled configuration B to the rolled configuration A. Thus, the cover 14 may be wrapped around the outer surface 28 of the longitudinal body 16 in at least one full 360 degree wrap (FIG. 1). Alternatively, the cover 14 may be wrapped around the outer surface 28 less than one full 360 degree wrap, in which case the second portion 34 would be removably coupled to the outer surface 28 of the longitudinal body 16.

With continued reference to FIG. 2, the main sheet 30 of the cover 14 includes information 38, which may be visible when the cover 14 is in the rolled configuration A and/or unrolled configuration B. For example, in the unrolled configuration B, the information 38 may be visible to a user on an inside surface 40 of the cover 14. The information 38 may pertain to at least one of dosage information, compound information, instructional information, and brand information.

In some aspects, the cover 14 includes additives for blocking ultraviolet rays from passing through the cover 14. This provides the advantage to prevent harm to the compound 26 when the cover 14 is in the rolled configuration A, which can be critical for maintaining the medical properties of the compound 26. In some aspects, the cover 14 includes brand information 42 on an outer surface 28 when in the rolled configuration A. Additionally, the plunger 24 may include information 44, which may be similar to the information 38 on the cover 14.

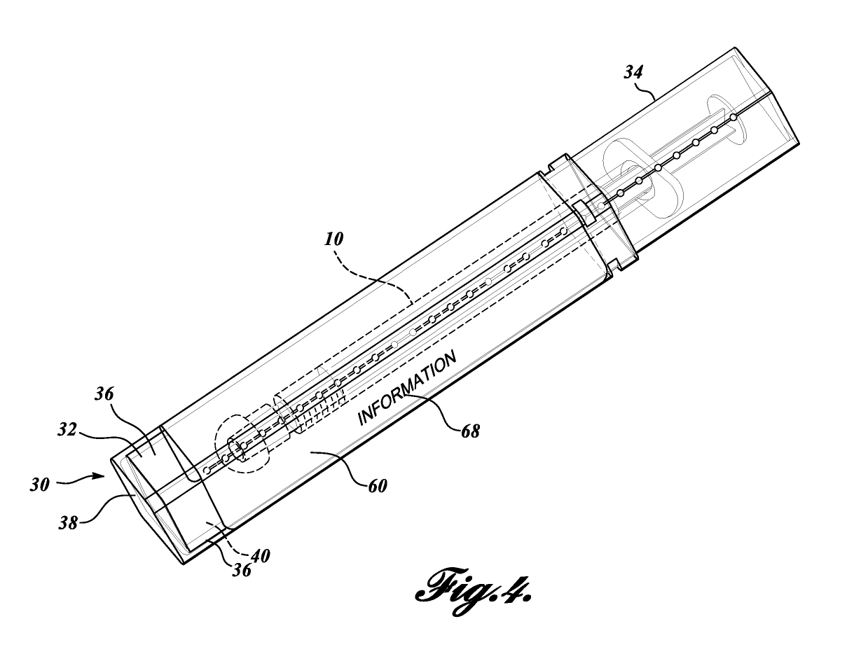

FIGS. 3 and 4 show a container assembly 30 that is adapted to hold therein a medical assembly 10, according to one embodiment. The container assembly 30 includes a first open-ended container 32 and a second open-ended container 34, which interface with one another to encapsulate the medical assembly 10. The first open-ended container 32 comprises four sidewalls 36 extending longitudinally from a bottom side 38 to define an internal chamber 40. An opening 42 is formed at one end of the four sidewalls 36, which end is opposite the bottom side 38. The opening 42 is sized and shaped to receive therethrough the medical assembly 10 and the second open-ended container 34 as discussed in more detail below. Proximal to the opening 42 and at edges where the sidewalls 36 intersect one another, the first open-ended container 32 includes grooves 44. The grooves 44 are formed on the outer surfaces of the sidewalls 36. Extending inwardly from the internal surfaces of the sidewalls 36 into the internal chamber 40 are projections 46, such as pegs or posts, or other engagement features.

The second open-ended container 34 comprises four sidewalls 48 extending longitudinally from a bottom side 50 to define an internal chamber 52. An opening 55 is formed at one end of the four sidewalls 48, which end is opposite the bottom side 50. The opening 55 is sized and shaped to receive therethrough the medical assembly 10. Along edges where the sidewalls 48 intersect one another, the second open-ended container 34 includes a plurality of recesses 56 spaced apart from one another. The recesses 56 are formed on the outer surfaces of the sidewalls 48. The recesses 56 are sized and shaped to engageably receive therein the projections 46 of the first open-ended container 32. In this manner, the first open-ended container 32 can slideably receive the second open-ended container 34. Although the embodiment illustrated in FIGS. 3-4 includes recesses 56 that are substantially spherically shaped, in other embodiments, the recesses 56 may be formed of any other shape, such as cubical, trapezoidal, or may include any other engagement feature.

Further, the plurality of recesses 56 are spaced apart from each other at distances that advantageously allow the container assembly 30 to receive therein a wide variety of medical assemblies 10 of varying lengths. By way of example, FIG. 4 illustrates a container assembly 30 in a closed configuration wherein the first open-ended container 32 overlies the second open-ended container 34. The first open-ended container 32 extends approximately three-quarter length of the second-open ended container 34 to encapsulate the medical assembly 10, thereby accommodating the medical assembly 10 of a certain length.

Similarly, where the medical assembly 10 is longer or shorter, the first open-ended container 32 may be moved further inwards or outwards relative to the bottom side 38 of the first open-ended container 32 to fully encapsulate the corresponding medical assembly 10.

Furthermore, the first and second open-ended containers 32, 34 both comprise bodies of transparent material, through which the contents thereof may be, at least, partially visible to a user. The first open-ended container 32 also includes an outer cover 60 that is coupled to the outer surfaces of the sidewalls 36. The outer cover 60 may be fully wrapped around the first open-ended container 32 or may be partially wrapped around the first open-ended container 32. The outer cover 60 may be coupled to the first open-ended container 32 via adhering or, in some embodiments, may be imprinted onto the cover using various imprinting techniques. The outer cover 60 includes information 68 that may be visible to the user. The information 68 may pertain to at least one of dosage information, compound information, instructional information, and brand information.

Moreover, the various embodiments described above can be combined to provide further embodiments. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A syringe, comprising:
a longitudinal body having an outer surface and a chamber, the chamber for holding a medical compound therein and to be dispensed by the syringe; and
a cover coupled to the longitudinal body and movable between a rolled configuration and an unrolled configuration, the cover having a first edge portion and a second edge portion, the first edge portion attached to the outer surface of the longitudinal body, the cover configured to be wrapped around the outer surface and the second edge portion configured to be removably coupled to a portion of the cover.

2. The syringe of claim 1 wherein the cover includes a main sheet extending between the first and second edge portions, the main sheet having information at least accessible when the cover is in the unrolled configuration.

3. The syringe of claim 2 wherein the information comprises at least one of dosage information, compound information, instructional information, and brand information.

4. The syringe of claim 1 wherein at least a portion of the cover is comprised of material adapted to prevent radiation from passing through the cover to prevent harm to the compound contained in the chamber.

5. The syringe of claim 1 wherein the first edge portion includes a perforated portion to remove the cover from the syringe.

6. The syringe of claim 1 wherein the second edge portion includes a re-sealable device configured to re-seal the cover to at least one of the syringe and the cover when the cover is moved from the unrolled configuration to the rolled configuration.

7. An assembly for medical use, comprising:
a syringe having a body, a chamber, a tip in fluid communication with the chamber, a medical compound inside the chamber, and a plunger member disposed at least partially within the chamber; and
a cover coupled to the body and movable between a rolled configuration and an unrolled configuration, the cover having a first edge, a second edge, and a sheet extending between the first and second edges, the first edge attached to the body and the sheet wrapped around the body, the second edge removably coupled to the sheet when the cover is moved to the rolled configuration, the sheet includes information pertaining to dosage of the medical compound.

8. The syringe of claim 7 wherein the first edge includes a perforated portion to remove the cover from the syringe.

9. The syringe of claim 7 wherein the information on the sheet comprises at least one of dosage information, medical compound information, instructional information, and brand information.

10. The syringe of claim 7 wherein at least a portion of the cover is comprised of material configured to prevent radiation from passing through the cover.

11. A container assembly, comprising:
a first open-ended container having a first internal chamber and a first engagement member;
a second open-ended container having a second internal chamber and a second engagement member, the second open-ended container sized and shaped to be slidably received in the first open-ended container, the second engagement member engaging the first engagement member to removably couple the first open-ended container to the second open-ended container; and
a medical assembly removably received in the first and second internal chambers of the respective first and second open-ended containers, the medical assembly comprising a syringe and a cover, the cover coupled to a body of the syringe and movable between a rolled configuration and an unrolled configuration, the cover having a first edge, a second edge, and a sheet extending between the first and second edges, the first edge attached to the body and the sheet wrapped around the body, the second edge removably coupled to the sheet when the cover is moved to the rolled configuration, the sheet including information.

12. The container assembly of claim 11, further comprising:
an outer cover at least partially wrapped around the first open-ended container, the outer cover including information pertaining to at least one of dosage information, compound information instructional information, and brand information.

13. The container assembly of claim 11 wherein the information comprises at least one of dosage information, compound information, instructional information, and brand information.

14. A method of making a medical assembly, comprising:
providing a syringe having a longitudinal body and a chamber, the longitudinal body having an outer surface and the chamber for holding a medical compound therein;
coupling a cover to the longitudinal body, the cover having a first edge portion and a second edge portion, the first edge portion attached to the outer surface of the longitudinal body;
wrapping the cover around the longitudinal body; and
removably attaching the second edge portion of the cover to an outer surface of the cover, the second edge portion having a re-sealable device to move the cover between a rolled configuration and an unrolled configuration.

15. The method of claim 14 comprising providing information on the cover that is at least accessible when the cover is in the unrolled configuration.

16. The method of claim 15 wherein the information comprises at least one of dosage information, compound information, instructional information, and brand information.

17. The method of claim 14 comprising fabricating the cover to have radiation blocking material to prevent harm to the compound contained in the chamber.

18. The method of claim 14 comprising perforating a portion of the cover to allow removal of the cover when in the unrolled configuration.

19. The method of claim 14 comprising attaching the re-sealable device to the second edge portion, wherein the re-sealable device is an adhesive.

20. The method of claim 14 comprising inserting the medical assembly into a container assembly for containing the syringe and the cover therein.
